(19) United States
(12) Patent Application Publication
(10) Pub. No.: US 2025/0244569 A1
(43) Pub. Date: Jul. 31, 2025

(54) BINOCULAR ASSEMBLY FOR USE WITH A MICROSCOPE

(71) Applicant: Alcon Inc., Fribourg (CH)

(72) Inventors: Carlos Vasseur, Huntington Beach, CA (US); Philip Matthew McCulloch, Oro Valley, AZ (US); Christopher Paul Voita, Dove Canyon, CA (US)

(73) Assignee: Alcon Inc., Fribourg (CH)

(21) Appl. No.: 19/035,218

(22) Filed: Jan. 23, 2025

Related U.S. Application Data
(60) Provisional application No. 63/625,078, filed on Jan. 25, 2024.

(51) Int. Cl. G02B 21/20

(52) U.S. Cl. CPC G02B 21/20

(57) ABSTRACT

A binocular assembly includes a first optical pathway extending from a flat plate mirror to a first eyepiece assembly. The first optical pathway includes a first penta-right angle prism, a first optical focusing assembly located along a first focusing axis for focusing the first optical pathway from the first penta-right angle prism, and a first pair of right-angle prisms located along a first prism axis. The binocular assembly also includes a second optical pathway extending from the flat plate mirror to a second eyepiece assembly. The second optical pathway includes a second penta-right angle prism, a second optical focusing assembly located along a second focusing axis for focusing the second optical pathway from the second penta-right angle prism, and a second pair of right-angle prisms located along a second prism axis.

### CROSS-REFERENCE TO RELATED APPLICATIONS

The present application claims the benefit of priority to U.S. Provisional Patent Application No. 63/625,078 filed on Jan. 25, 2024, which is hereby incorporated by reference in its entirety.

### INTRODUCTION

This disclosure relates generally to a binocular assembly for use with a microscope. More particularly, this disclosure relates to an optical train of a binocular assembly for use with a surgical microscope.

Ophthalmic surgery is performed on the eye and accessory visual structures. During ophthalmic surgery, a patient is placed on a support, facing upward, under a surgical microscope. Surgeons often use a surgical microscope to view the patient's eye, and surgical instruments may be introduced to perform any of a variety of different procedures. The surgical microscope provides imaging and optionally illumination for parts of the eye during the procedure. A surgical microscope may be configured in many forms, for example, as a ceiling-mounted surgical microscope or a mobile, cart-mounted surgical microscope.

### SUMMARY

Disclosed herein is a binocular assembly. The binocular assembly includes a first optical pathway extending from a flat plate mirror to a first eyepiece assembly. The first optical pathway includes a first penta-right angle prism, a first optical focusing assembly located along a first focusing axis for focusing the first optical pathway from the first penta-right angle prism, and a first pair of right-angle prisms located along a first prism axis for directing the first optical pathway from the first focusing axis to a first eyepiece axis. The first eyepiece assembly is located along the first eyepiece axis. A second optical pathway extends from the flat plate mirror to a second eyepiece assembly. The second optical pathway includes a second penta-right angle prism, a second optical focusing assembly located along a second focusing axis for focusing the second optical pathway from the second penta-right angle prism, and a second pair of right-angle prisms located along a second prism axis for directing the second optical pathway from the second focusing axis to a second eyepiece axis. The second eyepiece assembly is located along the second eyepiece axis.

In one embodiment, the first pair of right-angle prisms are each adjustably attached to a corresponding one of a first pair of flexure mounts.

In one embodiment, the first pair of flexure mounts include a first upper flexure mount that allows for adjustment about a first axis generally parallel to the first focusing axis and a second axis generally located at the midpoint of a first upper prism's hypotenuse of the first pair of right-angled prisms and generally perpendicular to a plane including the first focusing axis and a first arm axis and a first lower flexure mount that allows for adjustment about a fourth axis generally parallel to the first arm axis and a third axis generally located at the midpoint of a first lower prism's hypotenuse of the first pair of right-angled prisms and generally perpendicular to a plane including the first arm axis and the first eyepiece axis.

In one embodiment, the second pair of right-angle prisms are each adjustably attached to a corresponding one of a second pair of flexure mounts.

In one embodiment, the second pair of flexure mounts include a second upper flexure mount that allows for adjustment about a fifth axis generally parallel to the second focusing axis and a sixth axis generally located at the midpoint of a second upper prism's hypotenuse of the second pair of right-angled prisms and generally perpendicular to a plane including the second focusing axis and a second arm axis and a second lower flexure mount that allows for adjustment about an eighth axis generally parallel to the second arm axis and a seventh axis generally located at the midpoint of a second lower prism's hypotenuse of the second pair of right-angled prisms and generally perpendicular to a plane including the second arm axis and the second eyepiece axis.

In some embodiments, the first focusing axis is generally parallel to the first eyepiece axis and the second focusing axis is generally parallel to the second eyepiece axis.

In one embodiment, the first optical focusing assembly includes a first focusing lens and a second focusing lens, with at least one of the first focusing lens or the second focusing lens being movable along the first focusing axis.

In one embodiment, the second optical focusing assembly includes a first focusing lens and a second focusing lens, with at least one of the first focusing lens or the second focusing lens being movable along the second focusing axis.

In some embodiments, the first penta-right angle prism and the second penta-right angle prism increase a width between the first optical pathway and the second optical pathway and the first penta-right angle prism and the second penta-right angle prism each include a right-angle prism adjacent to a pentaprism.

In one embodiment, a lateral spacing of the first optical pathway entering the first penta-right angle prism from the second optical pathway entering the second penta-right angle prism is between about 18 mm (0.71 inches) and about 25 mm (0.98 inches).

In one embodiment, a lateral spacing of the first optical pathway exiting the first penta-right angle prism from the second optical pathway exiting the second penta-right angle prism is between about 58 mm (2.28 inches) and about 67 mm (2.64 inches).

In one embodiment, the first prism axis extends through a first arm with a proximal end of the first arm at least partially aligned with the first focusing axis and a distal end of the first arm at least partially aligned with the first eyepiece axis.

In one embodiment, the second prism axis extends through a second arm with a proximal end of the second arm at least partially aligned with the second focusing axis and a distal end of the second arm at least partially aligned with the second eyepiece axis.

Disclosed herein is an ophthalmic surgical assembly. The assembly includes a magnifying lens and a binocular assembly. The binocular assembly includes a first optical pathway extending from a flat plate mirror to a first eyepiece assembly. The first optical pathway includes a first penta-right angle prism, a first optical focusing assembly located along a first focusing axis for focusing the first optical pathway from the first penta-right angle prism, and a first pair of right-angle prisms located along a first prism axis for directing the first optical pathway from the first focusing axis to a first eyepiece axis. The first eyepiece assembly is located along the first eyepiece axis. A second optical pathway extends from the flat plate mirror to a second eyepiece assembly. The second optical pathway includes a second penta-right angle prism, a second optical focusing assembly located along a second focusing axis for focusing the second optical pathway from the second penta-right angle prism, and a second pair of right-angle prisms located along a second prism axis for directing the second optical pathway from the second focusing axis to a second eyepiece axis. The second eyepiece assembly is located along the second eyepiece axis.

In one embodiment, the binocular assembly is fixed relative to a surgical robot.

In one embodiment, the first optical focusing assembly includes a first focusing lens and a second focusing lens, with at least one of the first focusing lens or the second focusing lens being movable along the first focusing axis.

In one embodiment, the second optical focusing assembly includes a first focusing lens and a second focusing lens, with at least one of the first focusing lens or the second focusing lens being movable along the second focusing axis.

In one embodiment, the first penta-right angle prism and the second penta-right angle prism increase a width between the first optical pathway and the second optical pathway with the first penta-right angle prism and the second penta-right angle prism each including a right-angle prism adjacent to a pentaprism.

### BRIEF DESCRIPTION OF THE DRAWINGS

FIG. 1 illustrates a representative operating room setup with a microscope having an example binocular assembly.

FIG. 2 illustrates a perspective view of the binocular assembly of FIG. 1.

FIG. 3 illustrates a schematic perspective view of a pair of optical pathways through the binocular assembly of FIG. 2.

FIG. 4 illustrates a side schematic view of a first optical pathway of the pair of optical pathways through the binocular assembly of FIG. 2.

FIG. 5 illustrates a side schematic view of a second optical pathway of the pair of optical pathways through the binocular assembly of FIG. 2.

Some embodiments of the present disclosure are now described, by way of example only, and with reference to the accompanying drawings. The same reference number represents the same element or the same type of element on all drawings.

### DETAILED DESCRIPTION

Embodiments of the present disclosure are described herein. It is to be understood, however, that the disclosed embodiments are merely examples and other embodiments can take various and alternative forms. The figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “upper,” “lower,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

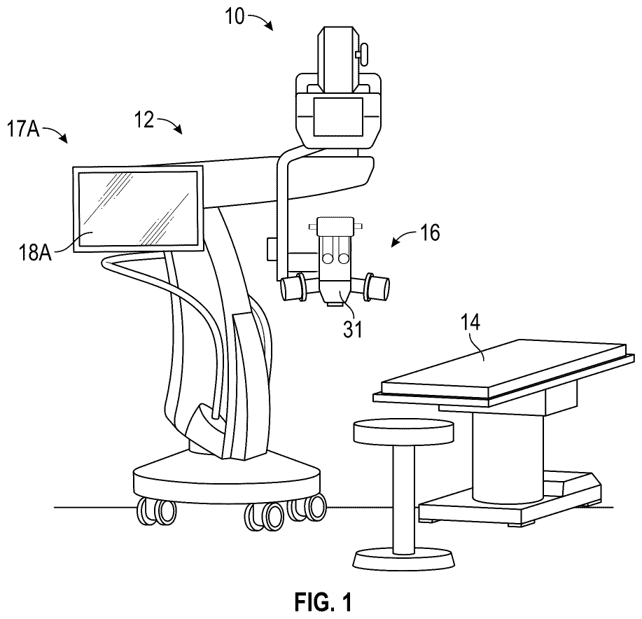

Referring to the drawings, wherein like reference numbers refer to like components, an ophthalmic surgical suite 10 is depicted schematically in FIG. 1. The range of possible ophthalmic procedures that could be performed within the ophthalmic surgical suite 10 may vary within the scope of the present disclosure. As appreciated by those skilled in the art, the ophthalmic surgical suite 10 of FIG. 1 may be equipped with a multi-axis surgical robot 12 and an operating table 14. The surgical robot 12 is typically connected to an ophthalmic grade microscope 16 through which a surgeon (not shown) is able to view a surgical field. For example, using associated hardware and software, the surgeon is able to view highly magnified images 18A, e.g., of a surgical field of view on screen 17A, respectively.

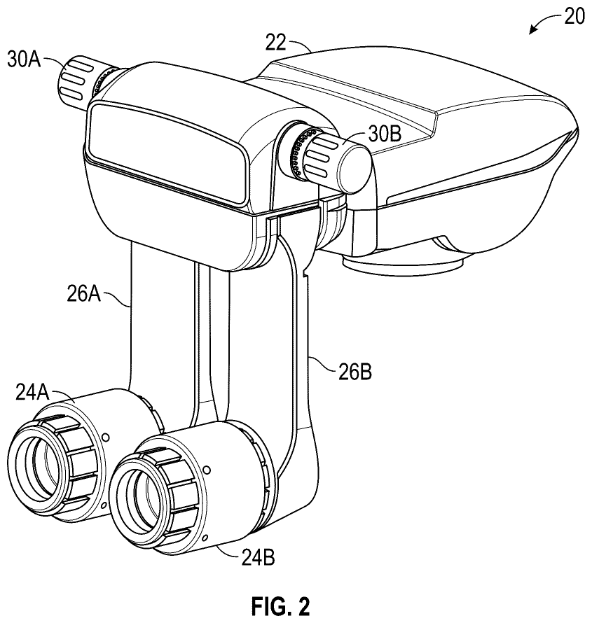

FIG. 2 illustrates first and second eyepieces 24A, 24B, for an example implementation of a binocular assembly 20 that forms a portion of the microscope 16 shown in FIG. 1. The binocular assembly 20 can be any suitable surgical binocular configured for use during an ophthalmic procedure. The binocular assembly 20 includes a housing 22 with first and second arms 26A, 26B extending from the housing 22 that support first and second eyepiece assemblies 24A, 24B, respectively. The first and second eyepiece assemblies 24A, 24B are attached to distal ends of the first and second arms 26A, 26B, respectively to allow the surgeon to view the surgical field at a reduced height. One feature of having the first and second eyepieces 24A, 24B located at distal end of the first and second arms 26A, 26B, respectively, is improved comfort when performing a procedure that would otherwise require the microscope 16 to be positioned at a height that may be uncomfortable to reach by some surgeons.

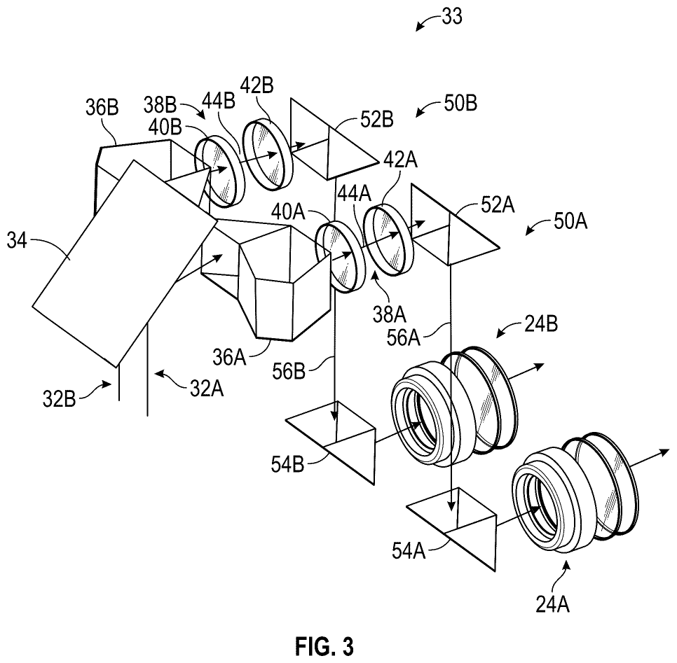

FIGS. 3-5 illustrate first and second optical pathways 32A, 32B for viewing light reflected from the surgical field that travels through the binocular assembly 20 to the first and second eyepieces 24A, 24B, respectively. In the illustrated example, the binocular assembly 20 is a stereo binocular assembly with the first and second optical pathways 32A, 32B providing separate view of the surgical field. The binocular assembly 20 includes one or more optical components, including lenses, mirrors, filters, gratings, prisms, and other elements that form an optical train 33 (FIG. 3) to allow the surgeon to view the surgical field. The optical train 33 may also include optical components for directing the light reflected from the surgical field into the first and second optical pathways 32A, 32B, such as a magnifying lens 31 (FIG. 1) attached to the binocular assembly 20.

In the illustrated example, light from the surgical field enters the binocular assembly 20 and is directed by a flat plate mirror 34. The first optical pathway 32A includes a first point of intersection with the flat plate mirror 34 that is offset from a second point of intersection of the second optical pathway 32B with the flat plate mirror 34. In the illustrated example, the first point of intersection is laterally offset by about 18-25 mm (0.71-0.98 inches) from the second point of intersection.

From the flat plate mirror 34, the first and second optical pathways 32A, 32B travel to a first and second penta-right angle prisms 36A, 36B, respectively. The first and second penta-right angle prisms 36A, 36B each include a right-angle prism that turns the light from the first flat plate mirror 34 by 90 degrees outward into a pentaprism. In the illustrated example, the pentaprism includes five sides separating an upper and a lower surface. A side of the pentaprism adjacent to the right-angle prism receives the light traveling along the optical pathways and reflects the light on two sides of the pentaprism before the light exits the pentaprism. The light enters a side of the pentaprism that is adjacent to a side of the prism where the light exits.

The lateral offset of the first and second optical pathways 32A, 32B prior to entering the first and second penta-right angle prisms 36A, 36B is increased upon exiting. In the illustrated example, the lateral offset for the first and second optical pathways 32A, 32B exiting the first and second penta-right angle prisms 36A, 36B, respectively, increases to about 58-67 mm (2.28-2.64 inches) of lateral offset. One feature of the increased lateral offset for the first and second optical pathways 32A, 32B is having an offset that more closely aligns with the spacing of the surgeon's eyes.

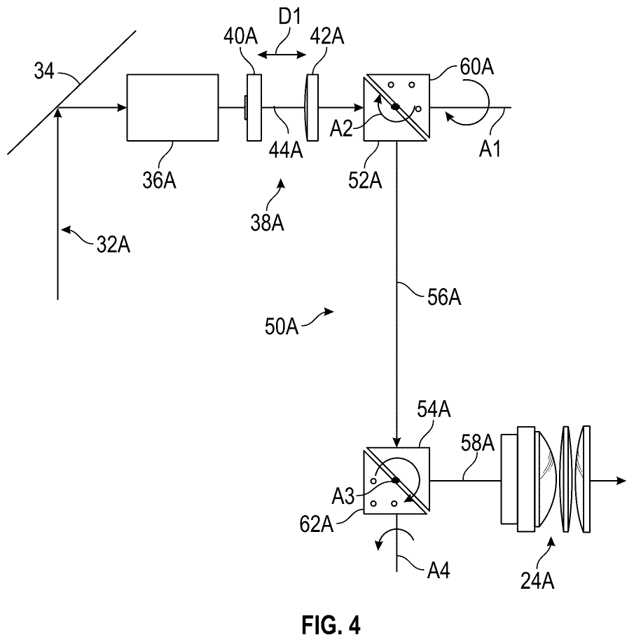

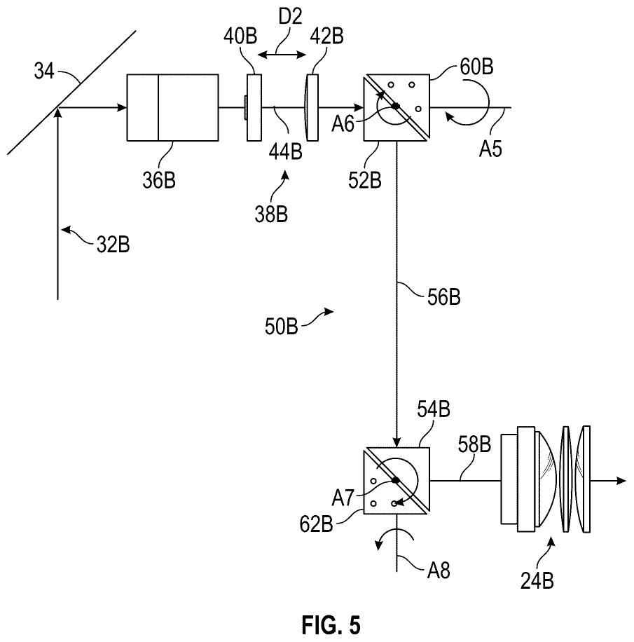

Once the light along the first and second optical pathways 32A, 32B has traveled through the first and second penta-right angle prisms 36A, 36B, the light is received by first and second optical focusing assemblies 38A, 38B, respectively. The first and second optical focusing assemblies 38A, 38B each include a first lens 40A, 40B and a second lens 42A, 42B, respectively. A distance D1 (FIG. 4), D2 (FIG. 5) between the first lens 40A, 40B and the second lens 42A, 42B varies to focus the first and second optical pathways 32A, 32B, respectively, to provide a clear view of the surgical field for the surgeon through the first and second eyepieces 24A, 24B. The lateral distance between first and second eyepieces 24A and 24B is adjustable by the surgeon with first and second adjuster knobs 30A, 30B, respectively, as shown in FIG. 2. The light traveling through the first and second optical focusing assemblies 38A, 38B travels along first and second focusing axes 44A, 44B, respectively. The first and second focusing axes 44A, 44B are each central longitudinal axes that extend from the first and second penta-right angle prisms 36A, 36B and through the first and second optical focusing assemblies 38A, 38B, respectively.

From the first and second optical focusing assemblies 38A, 38B, light traveling along the first and second optical pathways 32A, 32B reaches first and second pairs of right-angle prisms 50A, 50B, respectively. The first and second set of right-angle prisms 50A, 50B each include upper prisms 52A, 52B and lower prisms 54A, 54B, respectively. The upper prisms 52A, 52B receive light along the first and second optical pathways 32A, 32B traveling along the first and second focusing axes 44A, 44B and turn the first and second optical pathways 32A, 32B each 90 degrees to direct the first and second optical pathways 32A, 32B along first and second arm axes 56A, 56B, respectively. From the first and second arm axes 56A, 56B, the first and second optical pathways 32A, 32B turn 90 degrees at the lower prisms 54A, 54B towards the first and second eyepieces 24A, 24B, respectively.

As shown in FIG. 4, the upper right-angle prism 52A is adjustably mounted along the first optical pathway 32A by a first upper flexure mount 60A. The first upper flexure mount 60A allows the upper right-angle prism 52A to be adjusted about an axis A1 that is generally parallel to the axis 44A and an axis A2 that is generally located at the midpoint of the prism's hypotenuse and generally perpendicular to a plane including the first focusing axis 44A and the first arm axis 56A. The lower right-angle prism 54A is adjustably mounted along the first optical pathway 32A by a first lower flexure mount 62A. The first lower flexure mount 62A allows the lower right-angle prism 54A to be adjusted about an axis A4 that is generally parallel to the axis 56A and an axis A3 that is generally located at the midpoint of the prism's hypotenuse and generally perpendicular to a plane including the first arm axis 56A and the first eyepiece axis 58A. In this disclosure, generally parallel is within optical tolerances of a person of ordinary skill in the art when aligning optical pathways.

As shown in FIG. 5, the upper right-angle prism 52B is adjustably mounted along the second optical pathway 32B by a second upper flexure mount 60B. The second upper flexure mount 60B allows the upper right-angle prism 52B to be adjusted about an axis A5 that is generally parallel to the axis 44B and an axis A6 that is generally located at the midpoint of the prism's hypotenuse and generally perpendicular to a plane including the second focusing axis 44B and the second arm axis 56B. The lower right-angle prism 54B is adjustably mounted along the second optical pathway 32B by a second lower flexure mount 62B. The second lower flexure mount 62B allows the lower right-angle prism 54B to be adjusted about an axis A8 that is generally parallel to the axis 56B and an axis A7 that is generally located at the midpoint of the prism's hypotenuse and generally perpendicular to a plane including the second arm axis 56B and the second eyepiece axis 58B.

The first and second upper flexure mounts 60A, 60B and the first and second lower flexure mount 62A, 62B allow for adjustment of the first and second optical pathways 32A, 32B to ensure that they are properly aligned with the first and second eyepieces 24A, 24B, respectively.

The first and second optical pathways 32A, 32B travel from the lower prism 54A, 54B to the first and second eyepiece assemblies 24A, 24B along the first and second eyepiece axes, 58A, 58B, respectively. The first and second eyepiece assemblies 24A, 24B each include a plurality of lens that receive light traveling from the lower right-angle prisms 54A, 54B, respectively, along the first and second optical pathways 32A, 32B.

One feature of the binocular assembly 20 is that the first optical pathway 32A follows a pathway that is a mirror image of the second optical pathway 32B. This improves consistency of the images viewed by the surgeon through the first and second eyepiece assemblies 24A, 24B. This also reduces the complexity and total number of individual components needed to build the optical train 33.

For purposes of this Detailed Description, unless specifically disclaimed: the singular includes the plural and vice versa; the words “and” and “or” shall be both conjunctive and disjunctive; the words “any” and “all” shall both mean “any and all”; and the words “including,” “containing,” “comprising,” “having,” and the like, shall each mean “including without limitation.” Moreover, words of approximation, such as “about,” “almost,” “substantially,” “generally,” “approximately,” and the like, may each be used herein to denote “at, near, or nearly at,” or “within 0-5% of,” or “within acceptable manufacturing tolerances,” or any logical combination thereof, for example. Lastly, directional adjectives and adverbs, such as fore, aft, inboard, outboard, starboard, port, vertical, horizontal, upward, downward, front, back, left, right, etc., may be with respect to a user standing on a horizontal surface.

While various embodiments have been described, the description is intended to be exemplary rather than limiting. It will be apparent to those of ordinary skill in the art that many more embodiments and implementations are possible that are within the scope of the embodiments. Any feature of any embodiment may be used in combination with or substituted for any other feature or element in any other embodiment unless specifically restricted. Accordingly, the embodiments are not to be restricted except in light of the attached claims and their equivalents. Also, various modifications and changes may be made within the scope of the attached claims.

What is claimed is:

1. A binocular assembly comprising:
a first optical pathway extending from a flat plate mirror to a first eyepiece assembly, the first optical pathway includes:
a first penta-right angle prism;
a first optical focusing assembly located along a first focusing axis for focusing the first optical pathway from the first penta-right angle prism;
a first pair of right-angle prisms located along a first prism axis for directing the first optical pathway from the first focusing axis to a first eyepiece axis, wherein the first eyepiece assembly is located along the first eyepiece axis; and
a second optical pathway extending from the flat plate mirror to a second eyepiece assembly, the second optical pathway includes:
a second penta-right angle prism;
a second optical focusing assembly located along a second focusing axis for focusing the second optical pathway from the second penta-right angle prism; and
a second pair of right-angle prisms located along a second prism axis for directing the second optical pathway from the second focusing axis to a second eyepiece axis, wherein the second eyepiece assembly is located along the second eyepiece axis.

2. The assembly of claim 1, wherein the first pair of right-angle prisms are each adjustably attached to a corresponding one of a first pair of flexure mounts.

3. The assembly of claim 2, wherein the first pair of flexure mounts include a first upper flexure mount that allows for adjustment about a first axis generally parallel to the first focusing axis and a second axis generally located at a midpoint of a first upper prism's hypotenuse of the first pair of right-angled prisms and generally perpendicular to a plane including the first focusing axis and a first arm axis and a first lower flexure mount that allows for adjustment about a fourth axis generally parallel to the first arm axis and a third axis generally located at the midpoint of a first lower prism's hypotenuse of the first pair of right-angled prisms and generally perpendicular to a plane including the first arm axis and the first eyepiece axis.

4. The assembly of claim 2, wherein the second pair of right-angle prisms are each adjustably attached to a corresponding one of a second pair of flexure mounts.

5. The assembly of claim 4, wherein the second pair of flexure mounts include a second upper flexure mount that allows for adjustment about a fifth axis generally parallel to the second focusing axis and a sixth axis generally located at a midpoint of a second upper prism's hypotenuse of the second pair of right-angled prisms and generally perpendicular to a plane including the second focusing axis and a second arm axis and a second lower flexure mount that allows for adjustment about an eighth axis generally parallel to the second arm axis and a seventh axis generally located at a midpoint of a second lower prism's hypotenuse of the second pair of right-angled prisms and generally perpendicular to a plane including the second arm axis and the second eyepiece axis.

6. The assembly of claim 1, wherein the first focusing axis is generally parallel to the first eyepiece axis.

7. The assembly of claim 1, wherein the second focusing axis is generally parallel to the second eyepiece axis.

8. The assembly of claim 1, wherein the first optical focusing assembly includes a first focusing lens and a second focusing lens, with at least one of the first focusing lens or the second focusing lens being movable along the first focusing axis.

9. The assembly of claim 1, wherein the second optical focusing assembly includes a first focusing lens and a second focusing lens, with at least one of the first focusing lens or the second focusing lens being movable along the second focusing axis.

10. The assembly of claim 1, wherein the first penta-right angle prism and the second penta-right angle prism increase a width between the first optical pathway and the second optical pathway.

11. The assembly of claim 10, wherein the first penta-right angle prism and the second penta-right angle prism each include a right-angle prism adjacent to a pentaprism.

12. The assembly of claim 1, wherein a lateral spacing of the first optical pathway entering the first penta-right angle prism from the second optical pathway entering the second penta-right angle prism is between about 18 mm (0.71 inches) and about 25 mm (0.98 inches).

13. The assembly of claim 1, wherein a lateral spacing of the first optical pathway exiting the first penta-right angle prism from the second optical pathway exiting the second penta-right angle prism is between about 58 mm (2.28 inches) and about 67 mm (2.64 inches).

14. The assembly of claim 1, wherein the first prism axis extends through a first arm with a proximal end of the first arm at least partially aligned with the first focusing axis and a distal end of the first arm at least partially aligned with the first eyepiece axis.

15. The assembly of claim 1, wherein the second prism axis extends through a second arm with a proximal end of the second arm at least partially aligned with the second focusing axis and a distal end of the second arm at least partially aligned with the second eyepiece axis.

16. An ophthalmic surgical assembly comprising:
a magnifying lens;
a binocular assembly attached to the magnifying lens, the binocular assembly comprising:
a first optical pathway extending from a flat plate mirror to a first eyepiece assembly, the first optical pathway includes:
a first penta-right angle prism;
a first optical focusing assembly located along a first focusing axis for focusing the first optical pathway from the first penta-right angle prism;
a first pair of right-angle prisms located along a first prism axis for directing the first optical pathway from the first focusing axis to a first eyepiece axis, wherein the first eyepiece assembly is located along the first eyepiece axis; and
a second optical pathway extending from the flat plate mirror to a second eyepiece assembly, the second optical pathway includes:
a second penta-right angle prism;
a second optical focusing assembly located along a second focusing axis for focusing the second optical pathway from the second penta-right angle prism; and
a second pair of right-angle prisms located along a second prism axis for directing the second optical pathway from the second focusing axis to a second eyepiece axis, wherein the second eyepiece assembly is located along the second eyepiece axis.

17. The assembly of claim 16, wherein the binocular assembly is fixed relative to a surgical robot.

18. The assembly of claim 16, wherein the first optical focusing assembly includes a first focusing lens and a second focusing lens, with at least one of the first focusing lens or the second focusing lens being movable along the first focusing axis.

19. The assembly of claim 16, wherein the second optical focusing assembly includes a first focusing lens and a second focusing lens, with at least one of the first focusing lens or the second focusing lens being movable along the second focusing axis.

20. The assembly of claim 16, wherein the first penta-right angle prism and the second penta-right angle prism increase a width between the first optical pathway and the second optical pathway with the first penta-right angle prism and the second penta-right angle prism each including a right-angle prism adjacent to a pentaprism.
